(19) United States
(12) Patent Application Publication
(10) Pub. No.: US 2003/0149349 A1
(43) Pub. Date: Aug. 7, 2003

(54) Integral patch type electronic physiological sensor

(76) Inventor: Thomas P. Jensen, Boise, ID (US)

Correspondence Address: PEDERSEN & COMPANY, PLLC, P.O. BOX 2666, BOISE, ID, 83701, US

(21) Appl. No.: 10/325,449

(22) Filed: Dec. 18, 2002

Related U.S. Application Data
(60) Provisional application No. 60/341,913, filed on Dec. 18, 2001.

(51) Int. Cl. A61B 5/04; A61B 5/02; A61B 5/08; A61B 5/00; A61B 5/103; A61B 5/117

(52) U.S. Cl. 600/372; 600/483; 600/484; 600/529; 600/549; 600/587

(57) ABSTRACT

A “smart bandage” or “smart patch” incorporates neither elastic bands nor wired individual sensors into its construction for physiological monitoring, especially heart rate sensing methods. The patch has an electronic circuit and battery, sandwiched between layers of insulating material and cover plastics. On the skin contacting side of the assembly, a pair of conductive, adhesive gel pads are intended to be placed upon the torso or abdominal area of a human or other mammal in order to sense the heart-rate voltage differential across the sensors. The signals are amplified and filtered by the internal electronics. A microcontroller then converts the heart-rate data information into one of multiple data output formats, which are sent by radio data transmission to any variety of outside receiving equipment.

This application claims priority of provisional application Serial No. 60/341,913, entitled “Bandage or Patch Type Physiological Sensor,” filed Dec. 18, 2001, which is hereby incorporated herein by this reference.

### BACKGROUND OF THE INVENTION

1. Field of the Invention

Embodiments of the present invention relate to EKG, pulse, respiration and other electronic physiological and/or environmental sensors.

2. Related Art

As an introduction to the problems solved by the present invention, consider the development of various present-day EKG or heart-rate electronic sensors. Existing art related to the field of this invention require that: 1) the user wear one or more elastic bands to hold the sensor or sensors in place, such as with an athletic heart-rate monitor, or 2) as in the case of standard, clinical EKG hardware, individual sensors be bonded to the skin with adhesive, and which are individually wired to the associated electronic monitoring equipment.

A large number of heart-rate monitoring products are commercially available from manufacturers like Acumen, Cardiosport, Freestyle, Polar and others, which sense the electrical activity of the heart through electrocardiogram type electrodes mounted in a flexible chest band assembly which is attached by way of an elastic strap around the torso.

There are several shortcomings to the present day art of the heart rate monitoring chest band. The first is that it is a physical encumbrance to the exerciser. Users of chest bands typically complain about chafing, interference with clothing and general discomfort. In addition, a chest band only functions when good electrical contact is established to the skin. In the case of the typical commercial chest band, this contact is achieved only by pre-moistening with water or conductive gel or by sweating during vigorous exercise. During very vigorous activity such as jumping or mountain biking the chest-band will often slip down from the optimum pick-up location. The resultant erratic EKG readings are sent out via the transmitted signal and further result in an erratic display and erratic averaging or other calculations based upon the corrupted data.

A typical heart rate monitoring device is described in U.S. Pat. No. 4,409,983, where Albert claims a method of monitoring a heart beat, filtering and averaging such data, amplifying such data differentially, and then sending the data through a processor and on to a display.

Further, in U.S. Pat. No. 4,625,733, Saynajakangas claims a method of taking a heart beat and an ECG signal, amplifying the signals, generating a field, detecting the field with a receiver, amplifying that signal, and then converting the signal to computer data.

Another reference is Polar Electro OY Published PCT Application WO95/05578 which claims a method of detecting pulse signals and sending the signals on to a transmitter which transmits such signals on to a receiver.

Finally, in European Published Application 0650695A2, Birnbaum claims a method of detection of a pulse signal, transmission of that signal to a receiver, processing that signal, the calculation of a mean pulse value, and then alerting the person using the device when a predetermined mean pulse value is achieved.

### SUMMARY OF THE INVENTION

None of the relevant prior art describe or claim the integrated, self-contained and self-attachment features of the present invention. Also, none of the prior art is constructed as a self-contained, self-attached device that integrates other sensors together with heart-rate/EKG electronic sensors to measure respiration, temperature, and/or other physiological or environmental parameters.

The present invention is identified by the inventor as a “smart bandage” or “smart patch”, which requires neither elastic straps, bands nor wired individual sensors in its construction. The device is an easy to use, wireless, self-contained assembly that can be removed and re-applied as needed.

The device of the present invention contains all necessary electronic circuitry, including sensors, a battery or other power source, and a microcontroller and/or other programmable logic circuitry that perform measurement and processing of sensed data, where said processed data are subsequently stored internally and/or transmitted to other equipment by wireless means. Different embodiments of the invention may contain heart-rate only, EKG only, heart-rate plus respiration rate, skin temperature, air temperature, humidity, skin conductance level, and/or other types of sensors, together or separate, in all permutations.

These and other embodiments, aspects, advantages and features of the present invention will be set forth in part in the description, and in part will come to those skilled in the art by reference to the following description of the invention and referenced drawings, or by practice of the invention.

### BRIEF DESCRIPTION OF THE DRAWINGS

FIG. 1 is a schematic block diagram of an electronic circuit in another embodiment of the present invention.

FIG. 2 is a schematic diagram of an electronic circuit in an embodiment of the present invention.

FIG. 3 is a schematic block diagram of an electronic circuit in an embodiment of the present invention.

FIG. 4 is a schematic block diagram of a transmitter electronic circuit in an embodiment of the present invention.

FIG. 5 is an exploded, perspective view of the component parts in an embodiment of the present invention.

FIG. 6 is a series of three (3) views of an alternate means of attaching a heart signal sensor pads to the printed circuit assembly in an embodiment of the present invention.

FIG. 7 depicts a first, partial, cut-away side view of an embodiment of the invention showing the means of mechanical connection of component parts using a single flex circuit.

FIGS. 8 and 8A depict a second, partial, cut-away side view of an embodiment of the invention showing the means of mechanical connection of component parts using two flex circuits.

FIG. 9 is a top, perspective view of an embodiment of the present invention showing the components of a piezoelectric respiration sensor.

FIG. 9A is a perspective view of an embodiment of the present invention showing the mounting of a piezoelectric respiration sensor in the sensor assembly.

FIG. 10 is a schematic diagram of an electronic circuit of an embodiment of the present invention showing respiration signal conditioning.

FIG. 11 is a schematic diagram of an electronic circuit of an embodiment of the present invention incorporating a 3-axis accelerometer and amplifiers.

FIG. 12 is a perspective, exploded view of an embodiment of the present invention showing the mounting of an air temperature sensor.

### DETAILED DESCRIPTION OF THE INVENTION

It is the function of the present invention to either a) electrically detect the occurrence of R events in the QRS complex of the EKG signal, and/or b) continuously sample the EKG signal, perform signal processing and calculation upon the data contained within those signals, and provide a transmitted data signal for reception by any variety of different receivers. According to this invention, these components are incorporated into a self-contained assembly that adhesively mounts to the torso of a person or mammal.

The features of the present invention that contribute to its usefulness and novelty include its physical construction and method of use. It is intended, as will be illustrated in the description below, that the present invention act as a totally self-contained sensor that is thin, being less than 6 or 7 mm in thickness, low in mass and may be applied to the skin much like a bandage, with self-adhesive pads and sensor materials on the skin-contact side of the invention. No extra straps or other means of attachment are required, although design features may be optionally provided for mounting of such a strap, as a secondary means of supplemental attachment of the device.

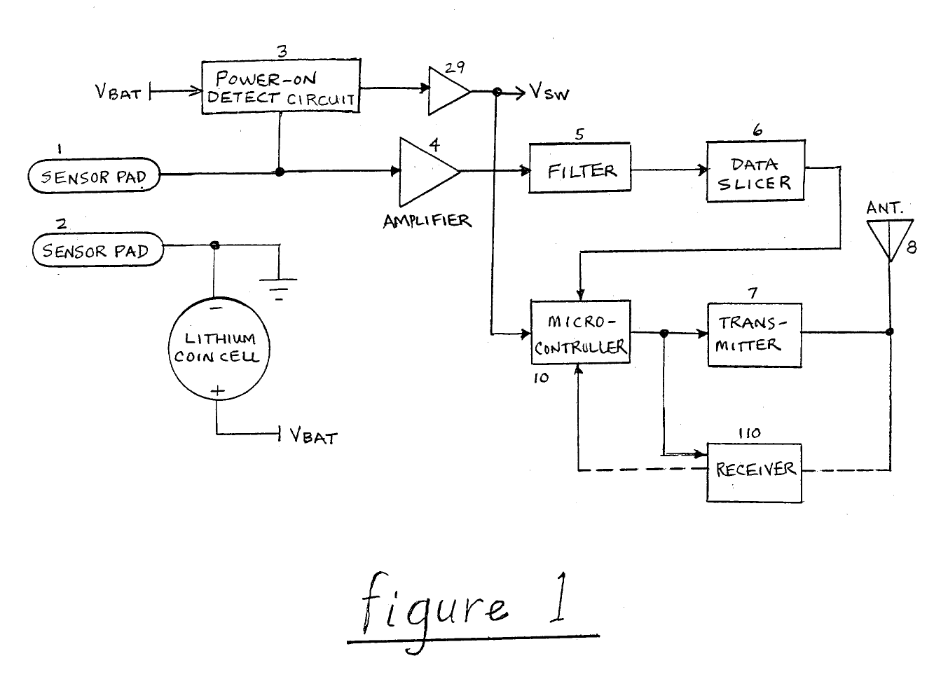

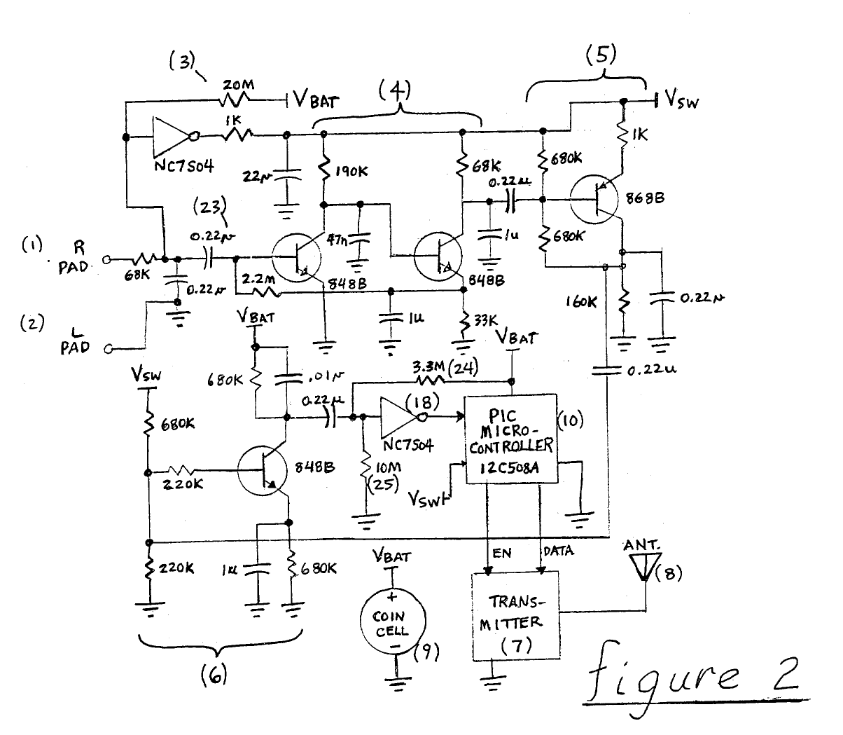

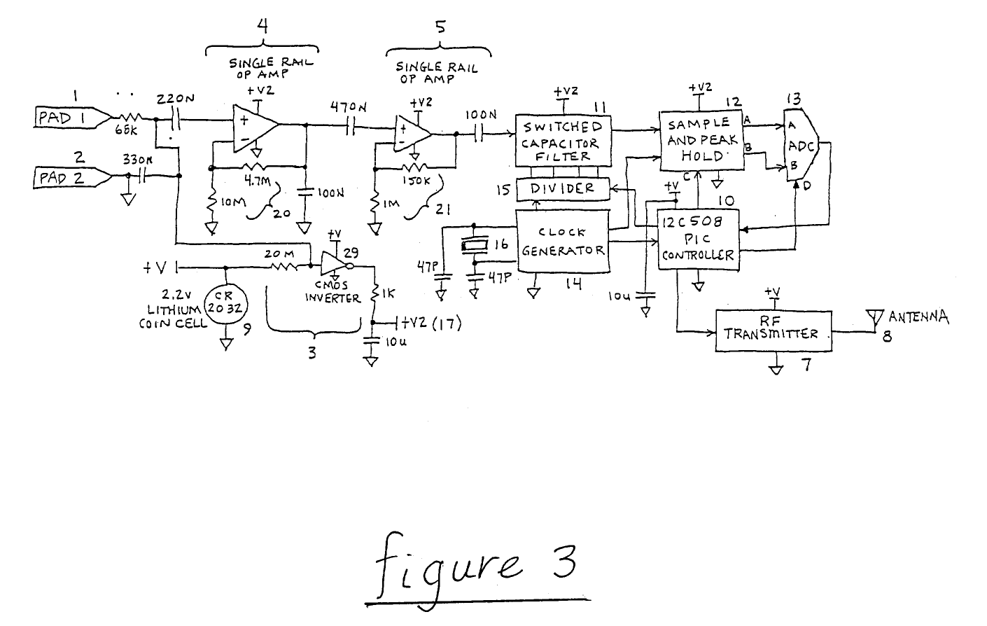

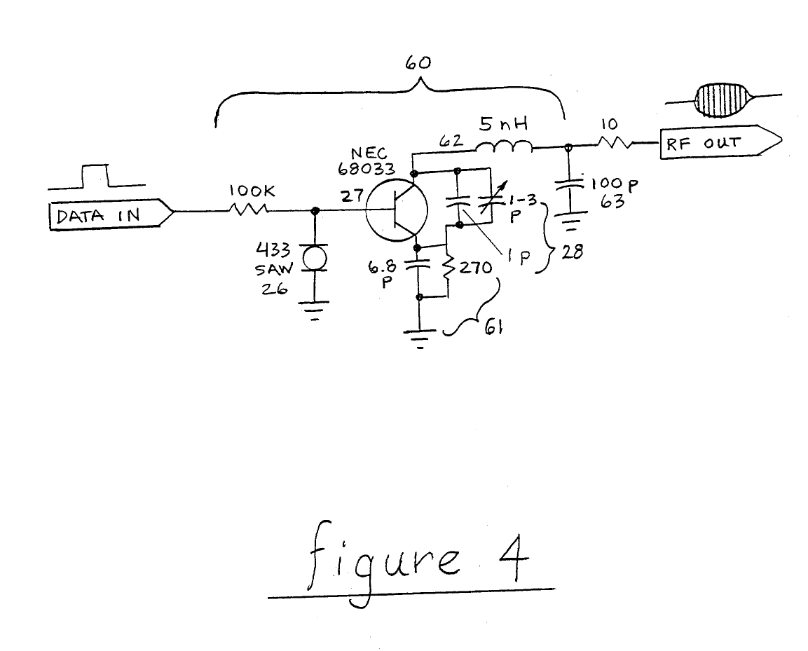

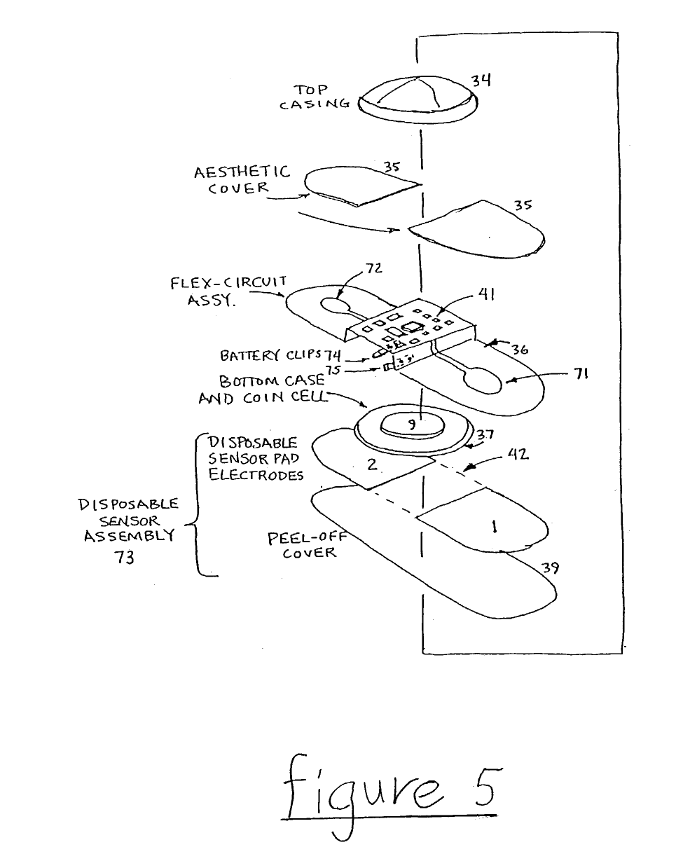

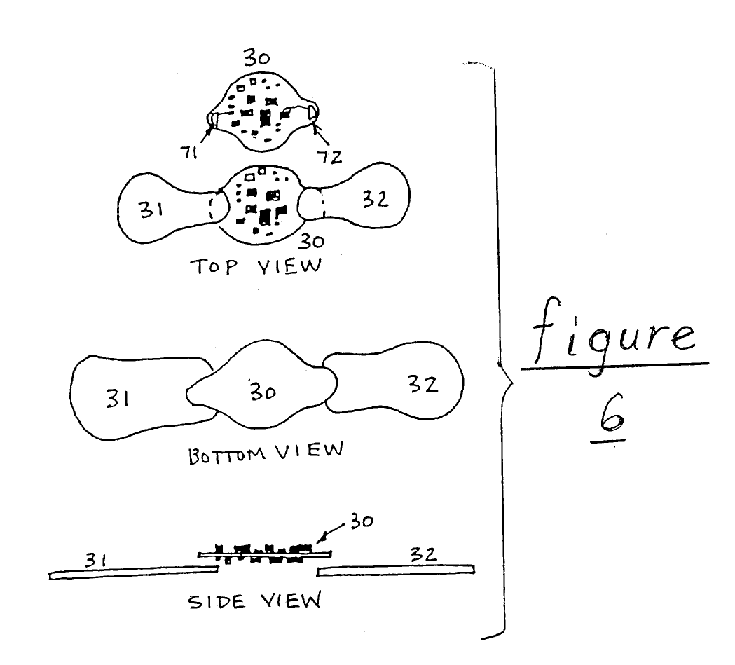

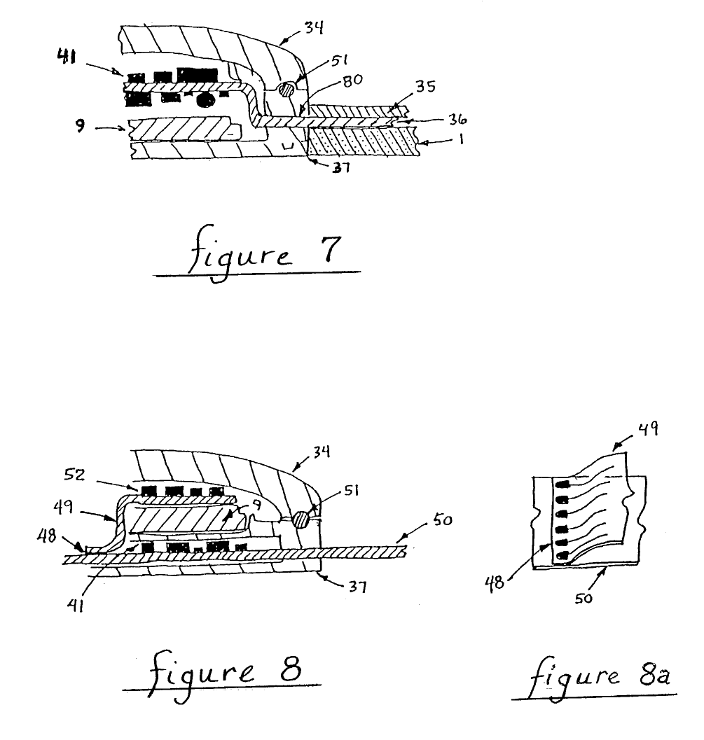

FIGS. 1 & 2 are schematic block diagrams of electronic circuits of two (2) different embodiments of the present invention. In these figures, heart-rate signals are collected from left and right sensor pads, 1 & 2. The signal from pad 2 is connected to the circuit ground, while the signal from pad 1 provides EKG signal input to a high gain amplifier 4 and acts as a triggering load for power-on detect circuit 3 once skin resistance is measured across the sensors. Logic driver 29, when enabled, supplies a switched supply voltage to disable and enable the operation of amplifier circuit section 4, filter circuit section 5 and data-slicer circuit section 6. With the use of switched supply voltage driver 29, power from the system power source 9 is conserved whenever the sensors 1, 2 are not in contact with the user's skin.

Signals from the sensors 1 & 2 are amplified by the amplifier circuit section 4. The amplified signal is then bandpass filtered by filter circuit section 5 and finally, signal extraction is accomplished by data-slicer circuit section 6. Signals that are output by the data-slicer circuit section 6 are further processed by components 24, 25 and 18 that make up the peak detector. Resistors 24 and 25 provide signal biasing, and driver 18 serves as a fixed threshold voltage comparator, allowing only signal levels that rise above the logic threshold to cause a logic shift at the driver 18 output. The resultant signal, at the output of driver 18, may either be input directly to transmitter 7, or may be input into microcontroller 10. Microcontroller 10 runs a conventional program that may perform further analysis and can also encode a data stream output to the transmitter 7.

In this way, two alternate methods are illustrated whereby transmitter data is either a) not encoded, but is sent as a single wave-pulse per heart-beat signal, or b) composed into an encoded data message and output by the program that that microcontroller 10 executes. Transmitter 7 may transmit in a variety of modulation and/or keying methods via antenna 8, especially when used in conjunction with microcontroller 10, whereby the microcontroller 10 may enable and disable the transmitter carrier, and also send encoded data streams. Data modulation methods in an RF transmitter that are easily implemented include the well understood methods of On-Off Keying (OOK), phase or frequency shift keying.

Rather than use an off-the-shelf microcontroller 10, the logic features could be implemented using another type of Programmable Logic Device (PLD) or a custom integrated circuit. However, these devices and circuits may be conventional ones. Other possible telemetry means include infrared mode transmission via IRLED, as intended to be received by a separate IR receiver device. Also, incorporation of an RF receiver 110 allows for reception of data from an outside source. An infrared receiver is an alternate data receiving option that has been practiced by this inventor for loading program code into a FLASH memory type of microcontroller 10. Such an infrared receiver can be used to import other data into the device as well, according, again, the conventional practices.

The existence of microcontroller 10 in the circuit also expands the possible additional physiological and environmental sensors that can be incorporated into the present invention. Since a microcontroller can typically have an integrated, multi-channel Analog to Digital Converter (ADC), it allows the inputting of many channels of sensor data. As it relates to alternate embodiments of the system of the present invention, microcontroller 10 has been used by the inventor, in addition to measuring heart rate, to incorporate measurement means for respiration, accelerometer, temperature and humidity, and to store and forward their measured data in the same manner as described above, using the Manchester encoding and the OOK modulation method. This same data could alternately be sent in numerous encoded formats via an infrared emitter, much like a commercial remote control device sends data.

FIG. 3 is a schematic block diagram of an electronic circuit in another embodiment of the present invention. The signal from heart-rate sensor pad 2 is connected to the circuit ground, while the signal from pad 1 is input to a high gain amplifier 4 and acts as a triggering load for power-on detect circuit 3 once skin resistance is measured on the sensors. Logic driver 29, when enabled, supplies a switched supply voltage +V2 (17) to disable and enable the operation of amplifier circuit section 4, filter circuit section 5, switched capacitor filter 11, peak sample and hold circuit 12, and comparator 13. Signals from the sensors 1, 2 are amplified by the amplifier circuit section 4, which is set to operate at a particular gain value by the gain setting resistors 20. The amplified signal is then bandpass filtered by filter circuit section 5, which is set to operate at a particular gain value by the gain setting resistors 21, and then fed into switched capacitor filter 11. Frequency divider 15 outputs filter control signals that modify the bandpass characteristics of filter 11. Divider 15 is controlled by signals from microcontroller 10. Clock generator 14 provides logic level clock signal outputs to microcontroller 10; sample and peak hold circuit 12 and divider circuit 15.

The output of Switched Capacitor Filter 11 is an amplified and filtered signal. The signal has bandpass cutoff frequencies of typically 1.5 Hz at the low-end and 17 Hz at the high-end. This signal is input to a sample and peak hold circuit 12, which has two outputs, as follows: (a) pass through, and (b) peak input signal which is held until being programmatically reset by microcontroller control line (c). Signals (a) and (b) are selected by the microcontroller 10 through input selector signal (d). The digital output of the ADC 13 is monitored by microcontroller 10 in a polling loop, or upon a timer interrupt. Once microcontroller 10 identifies the data as having a heart beat pulse, (a clearly identifiable waveform with a distinctive shape and a high signal amplitude compared to typical ambient noise from the sensor), then the program analyzes the data in a number of optional ways. For example, the program may determine the time interval since the last beat was detected. The microcontroller has the capability of performing conventional programmatic signal analysis to create and transmit different data records, as shown in Table 1 immediately following.

TABLE 1
Signal Processing Algorithms

| Signal Analysis | Description | Implications |
|---|---|---|
| Beat Reporting | Microcontroller simply | No data protocol |
| | sends transmission that | necessary. Simply a |
| | tells of the occurrence of a | transmission to say |
| | beat. | there was a beat. Can |
| | | be a packet or just |
| | | a carrier signal, |
| | | switched on for a |
| | | short period, |
| | | then off again. |
| Logged Event | Microcontroller sends a | Store and forward |
| Reporting | packet periodically | data handling yields |
| | containing a log of all data | system efficiencies |
| | points since last | |
| | transmission. | |
| Beat to Beat | Microcontroller sends data | Providing this beat |
| Interval (BBI) | that is the interval in | to beat interval info |
| | milliseconds since the last | simplifies data |
| | beat. | processing. |
| Rolling Average | Data packet contains the | Providing this |
| Heart-rate | rolling average of the | rolling average data |
| (RAHR) | heart-rate for the last n | processing in the |
| | beats. | transmitter simplifies |
| | | receiver's data |
| | | processing. |

The data listed in Table 1 may be sent separately or in combinations. For example, the beat-to-beat interval data may be sent together in a single packet with average heart-rate data. Further, there are conventional algorithms that may be executed within microcontroller 10 that may provide further signal acquisition and analysis of information to the receiving equipment. Examples of these include, but are not limited to respiration, acceleration, temperature and humidity data.

FIG. 4 is a schematic block diagram of a transmitter electronic circuit. This circuit has been demonstrated to successfully transmit OOK data in the present invention. This circuit includes an oscillator 60, which is comprised of surface acoustic wave (SAW) resonator 26, RF transistor 27, feedback capacitors 28 and tuned circuit components 61, 62 and 63. The RF output mirrors the logic waveform at the data input by producing an RF carrier that matches the specific resonant frequency of the resonator 26.

FIG. 5 is an exploded view of the comonent parts in an embodiment of the present invention. This figure depicts the physically assembled electronic components and circuits described above, incorporated into a wearable assembly. The assembly is comprised of a flexible circuit assembly 36 that contains the copper wiring traces to connect the entire circuit 41 to the sensor contacts 71, 72 and, for example, Lithium coin cell 9. Alternately, a rechargeable type of power source, or a solar cell, for example, may be incorporated.

The sensor contacts 71, 72 make electrical contact with sensor pads 1 and 2. In this embodiment, the disposable sensor pad electrodes 1 and 2 are coated with a conductive adhesive on the circuit side and a conductive adhesive-gel that is made using a silver amalgam as found in off-the-shelf EKG sensor pads, such as those sold by 3M Corporation. In alternate embodiments, this material may be composed of a conductive rubber or synthetic rubber of any reasonably specified durometer, or a treated, conductive open cell foam, or a conductive silicone material. Each of these may be surrounded by a skin-adhesive ring of material, with protective cover tape 39.

The pads, when they are first installed, typically come pre-applied to a peel-off cover 39 that protects the conductive surface of sensor pads 1 and 2 until ready for use. Sensor pads 1 and 2 can also have optional, non-conductive connecting material 42. This material 42, if incorporated, will be a part of the disposable sensor assembly 73, which is comprised of pads 1, 2, cover 39 and connective material 42. All of the information illustrated in FIG. 5 is intended to be exemplary. It should in no way limit other possible component or material choices nor possible construction methods that may be used to make a device that falls within the scope of this invention

The re-usable (non-disposable) portion of the invention is further comprised of bottom case 37, top casing 34 and two aesthetic covers 35. Bottom case 37 may be constructed of ABS or other suitable type of plastic. It provides a mounting position and backing for Lithium coin cell 9. Top casing 34 and two aesthetic covers 35 may be constructed from Mylar sheet, for example, and enclose the entire top side of the flexible circuit assembly 36. When fully assembled, the invention is sealed on top, and has a location on the bottom side for the disposable sensor assembly 73 to be applied. Lithium coin cell 9 attaches to flexible circuit assembly 36 with two small nickel or gold plated steel clips 74, 75. The entire assembly may be designed in such a way that it is easily assembled in a set of progressive operations whereby reels or rolls of die-cut cover materials, along with the pre-assembled electronics, are applied by machine operation to the inner circuitry and connections, yielding a final product that may be more easily mass-produced than if handling and hand operations were required.

FIG. 6 is a series of three (3) views of an alternate means of attaching the heart signal sensor pads to the printed circuit assembly. This method incorporates a printed circuit assembly 30, which is built upon a rigid or semi-rigid substrate with etched copper wiring traces. Disposable sensor pads 31 and 32 attach to sensor contacts 71, 72

FIG. 7 depicts a first, partial cut-away side view of an embodiment of the invention showing the means of mechanical connection of inter-related parts using a single flex circuit, such as the one depicted in FIG. 5. This view illustrates the sandwich type of construction of physical components surrounding the electronics circuit 41. Top case 34 mounts onto bottom case 37, which, in this embodiment, has a slot cut-out 80 for the passage of flexible circuit assembly 36 to pass through. A sealant is applied to the slot 80, once assembled, to seal the electronics 41 from the surrounding environment. The top and bottom case halves 34, 37 have an o-ring seal 51 at their mating surfaces. This view also depicts a cut-away view of the Lithium coin cell 9, one aesthetic cover 35 and one sensor pad 1

FIG. 8 depicts a second, partial cut-away side view of an embodiment of the invention showing the means of mechanical connection of inter-related circuits using two flexible printed circuits 49, 50. The presence of the second flexible circuit assembly 49, which is connected to flexible circuit 50 by means of a set of pads 48, is depicted in FIG. 8A. The pads 48 at the connection of flexible circuit 49 and 50 are matched on both circuits, with the matched pads of tinned-copper exposed on the bottom side of circuit 49 and the top side of circuit 50. The pads are heat re-flowed together, melting the tinning metal, to connect the two circuits. By constructing the invention using this detail, there is extra area on the two flexible circuits for additional electronics.

In an alternate embodiment of the present invention, a respiration sensor is implemented, in addition to the heart rate sensing means heretofore described. FIG. 9 is a top view of an embodiment of the present invention showing the components of a piezoelectric respiration sensor. Sensor 81 is constructed of insert type rivets or similar fasteners 85 that hold an elastic coupler 83 in tension with a Penwall Corp. Kynar TM piezoelectric strip 84. These components may be fastened with epoxy, cyanoacrylate or other suitable adhesive at glue joints 82. The leads 86 from the piezoelectric strip provide the electrical signal output.

FIG. 9a is a perspective view of an embodiment of the present invention showing the mounting of a piezoelectric respiration sensor in the sensor assembly. The left and right sensor pads, 1 and 2, are shown fastened to the piezoelectric sensor 81 via fasteners 85. The gap between left a right sensor pads, 1 and 2, allows independent movement of the pads, and the tension analog is measured by the strain on sensor strip 84.

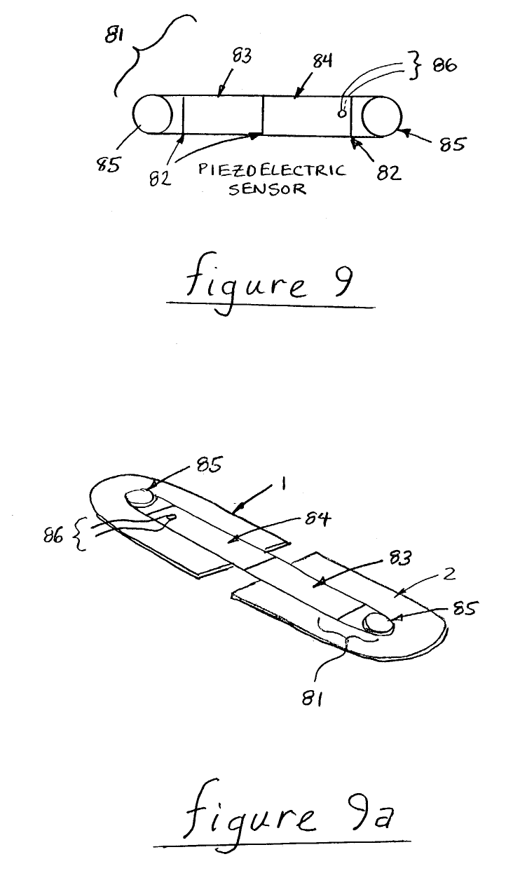

FIG. 10 is a schematic diagram of an electronic circuit of an embodiment of the present invention showing respiration signal conditioning. It shows sensor 81 being connected to the amplifier circuit 87 via leads 86. The output of the amplifier is input to a bandpass filter 89. Amplifier 87 provides a gain in the neighborhood of 900, and the bandpass filter has a −3 dB cutoff frequencies of typically 0.05 and 1.2 Hz. Feedback resistor 88 establishes front end gain. Similar other feedback correction circuits may provide other amplification effects, such as an auto-zeroing function. The output signal 90 is input to the microcontroller ADC 10 of FIGS. 1, 2, and 3.

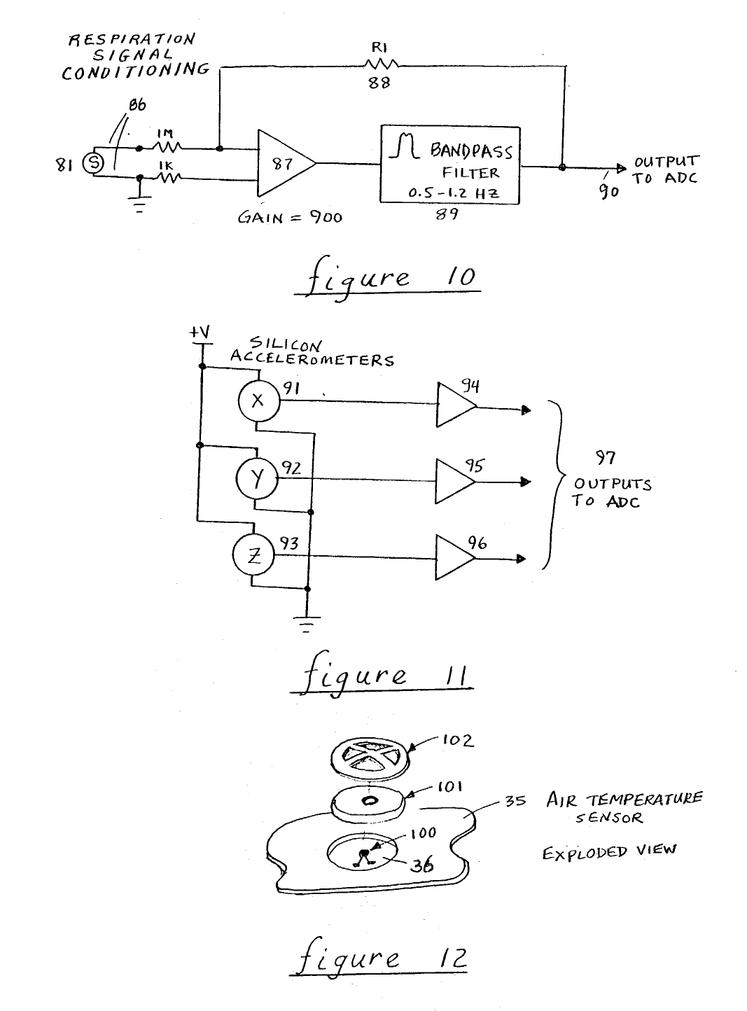

In an alternate embodiment of the present invention, one or more acceleration sensors may be implemented, in addition to the heart rate sensing means heretofore described. FIG. 11 is a schematic diagram of an electronic circuit of an embodiment of the present invention incorporating a 3-axis accelerometer and amplifiers. The three sensors 91, 92, and 93 are amplified by the three instrumentation amplifiers 94, 95 and 96, and their outputs 97 are input to the microcontroller ADC 10 of FIGS. 1, 2, and 3.

It should also be noted that such a sensor, using either the piezoelectric or the strain-gauge measurement means, may also be utilized for the detection of the heart beat by way of filtering and discerning the signature pressure or acoustical waves of a beat. These beat data may be detectable by a circuit much like the one in FIG. 10, if slightly different filter bandpass characteristics are used. The sensor may be placed over the sternum, jugular, aorta or other body locations where detection of the heart beat by way of pressure or acoustical waves may be achieved. Such a sensing capability in the device may replace or supplement the data gained by EKG sensing methods.

In an alternate embodiment of the present invention, one or more temperature sensors may be implemented, in addition to the heart rate sensing means heretofore described. One such temperature sensor may be applied as a skin temperature sensor. This may be easily implemented by exposing a temperature sensor such as the AD590 by Analog Devices to the skin, or to a thin skin-contact layer through which the skin temperature is thermally conducted. Another such temperature sensor may be implemented in such a way as to read air temperature.

FIG. 12 is a perspective exploded view of an embodiment of the present invention showing the mounting of an air temperature sensor in the present invention. Sensor bead 100, or other thermally isolated sensor elements is mounted on circuit assembly 36, which is visible in this view through an opening in cover 35. Foam insulator 101 further thermally isolates sensor 100 from the surrounding solid materials such that air temperature is the primary determinant of sensor temperature. Protective cover 102 allows airflow. The signal interface of such a temperature sensor may be commercially implemented using a variety of possible interface methods. Appropriate amplification means may be used, or in many cases the signal can be input directly into the microcontroller 10 of FIGS. 1, 2, and 3. The same may be said for sensing of other phenomena, such as relative humidity. For example, the Honeywell HIH-3605 humidity sensor may be interfaced directly with the ADC of microcontroller 10 to read relative humidity, and may be mounted at the top surface of the sensor.

The advantages of this invention for monitoring of heart-rate and other physiological and environmental data will be beneficial in the areas such as in sport, recreational, patient care and in military applications. Although this invention has been described above with reference to particular means, materials and embodiments, it is to be understood that the invention is not limited to these disclosed particulars, but extends to all equivalents within the field of this invention as established by the following claims.

What is claimed is:

1. A thin self-contained device capable of securement to the skin of a person or mammal, comprised of:
an adhesive area on a skin-contact side of the device, said adhesive area providing a means of attachment to the skin without a band or a strap,
two or more EKG sensing surfaces located on the skin-contact side of the device,
one or more electronics circuit assemblies, one or more of which includes a means for processing input signals, storing data and generating output signals, and one of which includes a transmitter circuit section, and,
a built-in power source.

2. A device in accordance with claim 1 that also incorporates:
a respiration sensor that outputs an electrical signal in response to expansion and contraction of the torso upon inhalation and exhalation,
a respiration signal conditioning circuit comprised of analog amplifier and/or filter sections, and
an analog to digital converter circuit for conversion of said electrical signal into a digital representation thereof.

3. A device in accordance with claim 1 that also incorporates:
a heart rate sensor that outputs an electrical signal in response to expansion and contraction of the body upon the pumping of blood through the heart, the aorta or an artery,
a heart rate signal conditioning circuit comprised of analog amplifier and/or filter sections, and
an analog to digital converter circuit for conversion of said electrical signal into a digital representation thereof.

4. A device in accordance with claim 1 that also incorporates:
one or more accelerometer sensors that output electrical signal(s) based upon accelerations in one or more axes,
one or more accelerometer analog amplifiers and/or filter sections, and
one or more analog to digital converter circuits for conversion of said electrical signal(s) into a digital representatio(s) thereof.

5. A device in accordance with claim 1 that also incorporates:
a skin temperature sensor that outputs an electrical signal, and
an analog to digital converter circuit for conversion of said electrical signal into a digital representation thereof.

6. A device in accordance with claim 1 that also incorporates:
an air temperature sensor that outputs an electrical signal, and,
an analog to digital converter circuit for conversion of said signal into a digital representation thereof.

7. A device in accordance with claim 1 that also incorporates:
an electronic humidity sensor that outputs an electrical signal,
a humidity signal conditioning circuit comprised of analog amplifier and/or filter sections, and
an analog to digital converter circuit for conversion of said electrical signal into a digital representation thereof.

8. A device in accordance with claim 1 that also incorporates an RF and/or infrared receiver, such that data may be received into as well as transmitted from said device.

9. A device in accordance with claim 1 that also incorporates a real-time clock, such that accurate time and date stamping of data may be a capability of said device.

10. A device in accordance with claim 1 that also incorporates mounting provisions for an optional elastic strap or band for secondary, supplemental means of attachment of the device to a person or animal.

11. A device in accordance with claim 5, which comprises an analog amplifier section for conditioning said electrical signal.

12. A device in accordance with claim 6, which comprises an analog amplifier section for conditional said electrical signal.
